(19) United States
(12) Patent Application Publication
(10) Pub. No.: US 2025/0339434 A1
(43) Pub. Date: Nov. 6, 2025

(54) TREATING NEUROLOGIC DISEASES

(71) Applicant: Richard Frye, Phoenix, AZ (US)

(72) Inventor: Richard Frye, Phoenix, AZ (US)

(21) Appl. No.: 18/653,409

(22) Filed: May 2, 2024

(51) Int. Cl. A61K 31/519; A61P 25/00; G01N 33/68

(52) U.S. Cl. CPC A61K 31/519; A61P 25/00; G01N 33/6896; G01N 2800/52

(57) ABSTRACT

A process for treating a human subject with a neurologic disorder comprises obtaining a sample of the human subject. The sample is contacted with an assay for detecting a presence of soluble folate binding protein (sFBP), one or more single nucleotide polymorphism (SNP) in folate or related one-carbon metabolism genes, or both. Based on the whether there is a presence of sFBP, a presence of SNPs, an amount of folate receptor alpha autoantibody (FRAA) (i.e., FRAA titer), or a combination thereof in the sample, a treatment including a folate is created. The treatment is then administered to the human subject.

### CROSS-REFERENCE TO RELATED APPLICATIONS

This application claims the benefit of U.S. Provisional Patent Application Ser. No. 63/499,515, filed May 2, 2023, entitled “TREATING NEUROLOGICAL DISEASES”, by Richard Frye, the disclosure of which is hereby incorporated by reference.

### BACKGROUND

Various aspects of the present invention relate generally to treating disorders and in particular to detecting and treating neurologic disorders in human subjects.

Neurologic disorders (including neurodevelopmental disorders, psychiatric disorders, etc.) affect many humans. For example, Autism Spectrum Disorder (ASD) affects 2% of children. However, it has been found that a folate receptor alpha autoantibody (FRAA), commonly found in individuals with ASD, disrupts folate transport across a blood-brain barrier into a brain through a folate receptor alpha transporter.

### BRIEF SUMMARY

According to aspects of the present disclosure, a process for treating a human subject with a neurologic disorder comprises obtaining a serum sample of the human subject. The serum sample is contacted with an assay for detecting a presence of soluble folate binding protein (sFBP), also known as the soluble folate receptor. Based on the whether there is a presence of sFBP in the sample, a treatment including a folate is created. The treatment is then administered to the human subject.

According to further aspects of the present disclosure, a process for treating a human subject with a neurologic disorder comprises obtaining a sample of the human subject. The sample is contacted with an assay for detecting one or more single nucleotide polymorphism (SNP) in folate and related one-carbon metabolism genes. Based on the whether there is a presence of the one or more SNP in the sample, a treatment including a folate is created. The treatment is then administered to the human subject.

According to still further aspects of the present disclosure, a process for treating a prenatal human subject with a potential for neurologic disorder comprises obtaining a sample of the prenatal human subject's gestational host. The sample is contacted with an assay for detecting a presence of soluble folate binding protein (sFBP), one or more single nucleotide polymorphism (SNP) in folate and related one-carbon metabolism genes, or both. Based on the whether there is a presence of sFBP, a presence of SNPs, an amount of folate receptor alpha autoantibody (FRAA) (i.e., FRAA titer), or a combination thereof in the sample, a treatment including a folate is created. The treatment is then administered to the gestational host of the human subject.

### BRIEF DESCRIPTION OF THE SEVERAL VIEWS OF THE DRAWINGS

FIG. 1 is a flow chart illustrating a first process for treating a human subject with a neurologic disorder, where the process uses the soluble folate binding protein to help determine a treatment, according to aspects of the present disclosure;

FIG. 2A is a chart illustrating results of the process of FIG. 1 when applied to twelve patients, where the results for a Total T-Score are shown on a Social Responsiveness Scale, according to various aspects of the present disclosure;

FIG. 2B is a chart illustrating results of the process of FIG. 1 when applied to twelve patients, where the results for an Awareness T-Score are shown on a Social Responsiveness Scale, according to various aspects of the present disclosure;

FIG. 2C is a chart illustrating results of the process of FIG. 1 when applied to twelve patients, where the results for a Cognition T-Score are shown on a Social Responsiveness Scale, according to various aspects of the present disclosure;

FIG. 2D is a chart illustrating results of the process of FIG. 1 when applied to twelve patients, where the results for a Communication T-Score are shown on a Social Responsiveness Scale, according to various aspects of the present disclosure;

FIG. 2E is a chart illustrating results of the process of FIG. 1 when applied to twelve patients, where the results for a Motivation T-Score are shown on a Social Responsiveness Scale, according to various aspects of the present disclosure;

FIG. 2F is a chart illustrating results of the process of FIG. 1 when applied to twelve patients, where the results for a Mannerisms T-Score are shown on a Social Responsiveness Scale, according to various aspects of the present disclosure;

FIG. 3A is a chart illustrating results of the process of FIG. 1 when applied to the twelve patients of FIGS. 2A-F, where the results for a Total raw score are shown on an Aberrant Behavior Checklist, according to various aspects of the present disclosure;

FIG. 3B is a chart illustrating results of the process of FIG. 1 when applied to the twelve patients of FIGS. 2A-F, where the results for an Irritability raw score are shown on an Aberrant Behavior Checklist, according to various aspects of the present disclosure;

FIG. 3C is a chart illustrating results of the process of FIG. 1 when applied to the twelve patients of FIGS. 2A-F, where the results for a Social Withdrawal raw score are shown on an Aberrant Behavior Checklist, according to various aspects of the present disclosure;

FIG. 3D is a chart illustrating results of the process of FIG. 1 when applied to the twelve patients of FIGS. 2A-F, where the results for an Stereotypy raw score are shown on an Aberrant Behavior Checklist, according to various aspects of the present disclosure;

FIG. 3E is a chart illustrating results of the process of FIG. 1 when applied to the twelve patients of FIGS. 2A-F, where the results for a Hyperactivity raw score are shown on an Aberrant Behavior Checklist, according to various aspects of the present disclosure;

FIG. 3F is a chart illustrating results of the process of FIG. 1 when applied to the twelve patients of FIGS. 2A-F, where the results for an Inappropriate Speech raw score are shown on an Aberrant Behavior Checklist, according to various aspects of the present disclosure;

FIG. 4 is a graph showing effects of a leucovorin treatment on patients with ASD, where the results are shown that the effects on a Social Responsiveness Scale is predicted by the amount of binding folate receptor alpha autoantibody (FRAA) in the blood (i.e., FRAA titer), according to various aspects of the present disclosure;

FIG. 5 is a is a flow chart illustrating a third process for treating a human subject with a neurologic disorder, where the process uses the level of the folate receptor alpha autoantibody binding protein to help determine a treatment, according to aspects of the present disclosure;

FIG. 6 is a flow chart illustrating a second process for treating a human subject with a neurologic disorder, where the process uses a certain single nucleotide polymorphism (SNP) in folate and related one-carbon metabolism genes to help determine a treatment, according to aspects of the present disclosure;

FIG. 7 is a is a flow chart illustrating a process for treating a prenatal human subject with a potential for a neurologic disorder, according to aspects of the present disclosure;

FIG. 8A is a chart illustrating the effect of a binding autoantibody titer on a human subject both with a folate and without a folate treatment, where the results for a Total T-score are shown on a Social Responsiveness Scale, according to various aspects of the present disclosure;

FIG. 8B is a chart illustrating the effect of a binding autoantibody titer on a human subject both with a folate and without a folate treatment, where the results for an Awareness T-score are shown on a Social Responsiveness Scale, according to various aspects of the present disclosure;

FIG. 8C is a chart illustrating the effect of a binding autoantibody titer on a human subject both with a folate and without a folate treatment, where the results for a Cognition T-score are shown on a Social Responsiveness Scale, according to various aspects of the present disclosure;

FIG. 8D is a chart illustrating the effect of a binding autoantibody titer on a human subject both with a folate and without a folate treatment, where the results for an Communication T-score are shown on a Social Responsiveness Scale, according to various aspects of the present disclosure; and

FIG. 8E is a chart illustrating the effect of a binding autoantibody titer on a human subject both with a folate and without a folate treatment, where the results for an SRS Motivation T-score are shown on a Social Responsiveness Scale, according to various aspects of the present disclosure.

### DETAILED DESCRIPTION

According to aspects of the present disclosure, neurodevelopmental disorders, neurobehavioral disorders, and psychiatric disorders (collectively referred to as “neurologic disorders” herein) biomarkers other than known biomarkers for the folate pathway, where the known biomarkers are the presence or absence of the folate receptor alpha autoantibody (FRAA) and variations in the methylenetetrahydrofolate reductase (MTHFR) gene (and in some cases, rare deleterious mutations in the folate receptor alpha gene). Using new biomarkers discussed herein instead of the known biomarkers or in conjunction with the known biomarkers provides a link to severity of the neurologic disorder in humans and helps prepare a treatment of folate for the human subject. Further, the new biomarkers discussed herein may be used in parents to guide prenatal and preconception treatment.

As the new biomarkers help determine a treatment (e.g., a dosage of leucovorin or another folate), they can help determine whether a patient is not responding an existing treatment because a dose is too low or because treatment with folate is inappropriate. As many physicians are reluctant to increase dosage beyond what is standard without a verified reason to do so, the treatment methods discussed herein offer choices to physicians and can help people suffering from certain neurological disorders.

The new biomarkers that can be used for treatment for neurological disorders include the FRAA titer, the soluble folate binding protein (sFBP) (which is also called a soluble folate receptor (sFR)) and a not uncommon single nucleotide polymorphism (SNP) in folate and related one-carbon metabolism genes, where a list of SNPs includes:
1. COMT 675 G>A;
2. MTR 2756 A>G;
3. MTHFR 677 C>T;
4. MTHFR 1298 T>G;
5. DHFR C>A;
6. RFC1 SLC19A1 80 A>G;
7. FOLR1 1816 delC;
8. FOLR1 1841 G>A;
9. TCN2 776 G>C;
10. CUBN G>A;
11. SHMT1 1420 C>T;
12. MTRR 66 A>G;
13. MTHFD2 A>G;
14. DNMT 3A T>C;
15. CTH 1208 G>T;
16. MTHFD2 T>C;
17. BHMT 742 G>A;
18. RFC1 80 A>G;
19. FOLH1 484 T>C; and
20. GCH1 G>A.

The SNPs above use a shorthand nomenclature of a name of a gene, a position within that gene, and then a change in the gene. For example, MTRR 66 A>G is the methionine synthase reductase, where an A to G substitution occurs at the 66th point in the nucleotide sequence (the official Human Genome Variation Society notation is MTRR c.66A>G). As another example, FOLR1 1816 delC means that in a folate receptor alpha gene, the C at position 1816 is deleted.

As mentioned above, FRAA titer, sFPB and SNPs may be used individually, in combination with one another, in combination with previously known biomarkers, or combinations thereof to help determine a treatment for neurological disorders.

Further, in a sample of twenty-one individuals with autism spectrum disorder (ASD) who were tested for FRAAs and SNPs above, it was found that the FOLR1 1816 delC SNP changes a magnitude of an effect of a binding FRAA on irritability as measured by an aberrant behavior checklist. For example, for each increase in one unit of the binding FRAA, irritability increased (worsened) 3.1 points more if a person was homozygous (SNP on each chromosome) for the FOLR1 1816 delC SNP. As another example, in a group of fifteen people, social motivation, as measured by the Social Responsiveness Scale, was worsened by 10.36 points for a one unit increase in the binding FRAA if the individual has at least one RFC1 80 A>G SNP.

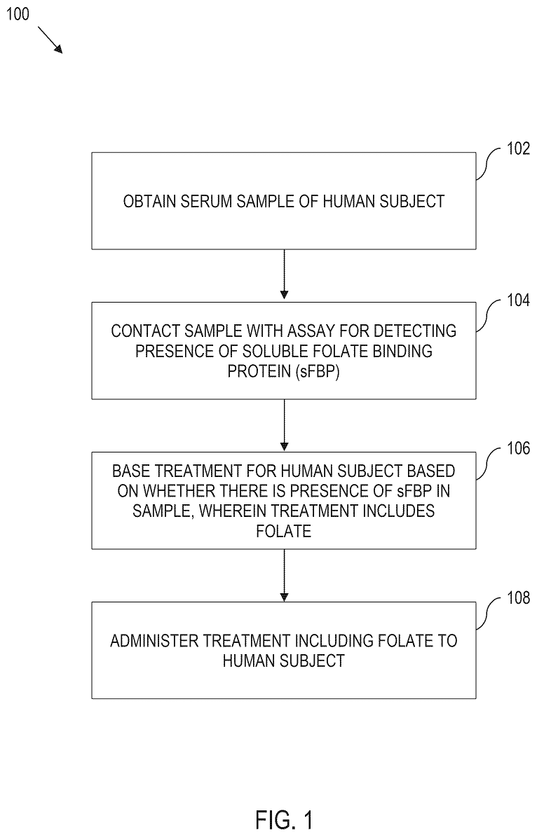

Turning now to the figures, and in particular FIG. 1, a process 100 for treating a human subject with a neurological disorder is shown. As discussed above, a neurologic disorder includes neurodevelopmental disorders, neurobehavioral disorders, and psychiatric disorders. For example, a neurologic disorder includes autism spectrum disorder (ASD). At 102, a serum sample is obtained from the human subject. Any method for obtaining the blood serum sample may be used (e.g., phlebotomy, receiving the serum sample previously drawn, etc.).

Once the serum sample is obtained, at 104, the serum sample is contacted with an assay for detecting a presence of soluble folate binding protein (sFBP). In some embodiments, the assay for detecting the presence of sFBP merely gives an indication of whether sFBP is present in the blood serum sample. In various embodiments, the assay for detecting the presence of sFBP gives an indication of whether sFBP present in the blood serum sample is above a predetermined threshold. In numerous embodiments, the assay for detecting the presence of sFBP detects a level of sFBP in the serum sample and gives an indication of the level of sFBP.

In several embodiments, the assay (or a different assay) also detects a presence of a folate receptor alpha autoantibody (FRAA) or a level of FRAA using a titer. As discussed above, the assay for detecting the FRAA may give an indication of a mere presence of FRAA, may give an indication of whether a level of FRAA is above a threshold, or may give an indication of a level of FRAA in the serum sample.

In many embodiments, the assay (or a different assay) also detects for a presence of a single nucleotide polymorphism (SNP) in folate or related one-carbon metabolism genes in the sample. As discussed above, the assay for detecting the SNP may give an indication of a mere presence of SNP, may give an indication of whether a level of SNP is above a threshold, or may give an indication of a level of SNP in the serum sample. In many embodiments, the assay can detect several different SNPs. For example, the assay can detect for RFC, FOLR1, BHMT, MTHFD2, DNMT, or combinations thereof. Thus, in these embodiments, five different SNPs may be used.

In two-hundred-and-eight subjects, communication abilities, as measured by the Vineland Adaptive Behavior Scale, was not only dependent on a presence of MTR 2756 A>G and MTHFR 1298 T>G SNPs but also an interaction between these two SNPs. For example, if an individual has no MTR 2756 SNPs, then there is minimal effect of the MTHFR 1298 SNPs on communication abilities, as shown in Table 1 below. Specifically, communication ability does not change much regardless of the number of MTHFR 1298 T>G SNPs if there are no MTR 2756 A>G SNPs. However, if an individual is homozygous (a SNP on each of the two chromosomes) for the MTR 2756 A>G SNP, then communication abilities decrease by 15.5 points if an individual has one (heterozygous) MTHFR 1298 T>G SNP and decreases by 20.1 points if an individual has two (homozygous) MTHFR 1298 T>G SNPs.

TABLE 1
Communication Ability Based on Number of SNPs
of Two Genes (higher score is better)

| | | MTHFR 1298 | | |
|---|---|---|---|---|
| Number of SNPs | | 0 | 1 | 2 |
| MTR2756 | 0 | 67.2 | 74.1 | 71.26 |
| | 1 | 75.3 | 64.3 | 59.8 |
| | 2 | 85.0 | 59.5 | 54.9 |

The results described above (i.e., communication abilities having differing results for the MTHFR 1298 T>G SNP based on a number of MTR 2756 A>G SNPs) was unexpected.

The three types of assays discussed above may be used in any combination. Further, one assay may detect sFBP, FRAA, and SNPs. On the other hand, three (or more) different assays may be used. Alternatively, one assay may detect two of the biomarkers and a second assay detects the third biomarker.

At 106, a treatment for the human subject is developed based on whether there is a presence of sFBP in the sample. A folate such as leucovorin (also called folinic acid) is used as at least part of the treatment. Leucovorin (folinic acid, Citrovorum factor) is a racemic mixture of the diastereoisomers of the 5-formyl derivative of tetrahydrofolic acid. The biologically active compound of the mixture is the (−)-L-isomer, known as Levoleucovorin. Leucovorin does not require reduction by the enzyme dihydrofolate reductase in order to participate in reactions utilizing folates as a source of “one-carbon” moieties. Following oral administration, leucovorin is rapidly absorbed and enters the general body pool of reduced folates. The increase in plasma and serum folate activity (determined microbiologically with Lactobacillus casei) seen after oral administration of leucovorin is predominantly due to 5-MTHF.

In certain embodiments, the reduced folate (drug) is folinic acid, 5-MTHF (5-methyltetrahydrofolate), and derivative, active metabolite, prodrug, stereoisomer, polymorph, analogue, or a pharmaceutically acceptable salt of any of the foregoing. In several embodiments, the reduced folate is D,L folinic acid calcium salt, or the pharmacologically active levo-isomer of d,l-leucovorin or a pharmaceutically acceptable salt thereof.

The folate may be in any desired form. However, at least one variable of the treatment is based on the sFBP found in the blood serum sample. For example, the treatment may include an X-dosage of folate given Y times per day with a time release of Z minutes. In embodiments that detect a level of sFBP in the serum sample, the dosage of folate may be linearly related (e.g., proportionally) to the level of sFBP, geometrically related to the level of sFBP, different level ranges may have different mathematical formulae, etc.

In embodiments that also look for SNPs and/or FRAA, the presence and/or levels of SNPs and/or FRAA also guide the variables for creating a treatment of folate for the human subject. For example, if a sample includes sFBP and FRAA (binding or blocking), then a higher dose of folate may be used. As another example, the titer of the binding FRAA (whether in conjunction with sFBP or not) may indicate a higher dosage. However, if MTHFD2 or DNMT (see list of SNPs above) is present, then the folate dosage may be lower. In several embodiments, mitochondrial supplements (e.g., carnitine) are added to the dosage of folate. In numerous embodiments, omega-3 fatty acids are added to the dosage of folate. The addition of mitochondrial supplements and/or omega-3 fatty acids have been shown to improve response of the human subject with respect to the treatment.

One of the objectives that the treatment may have is to enhance folate metabolism within the human subject.

At 108, the treatment is administered to the human subject.

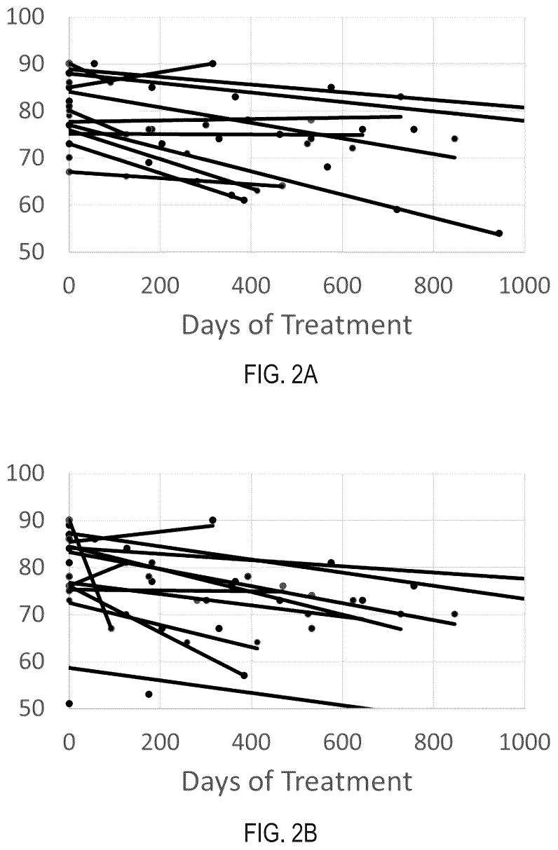

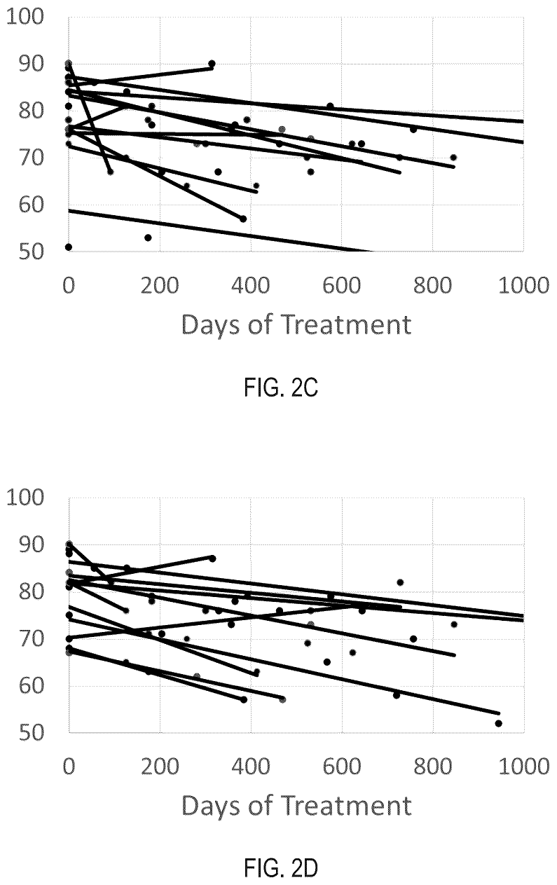

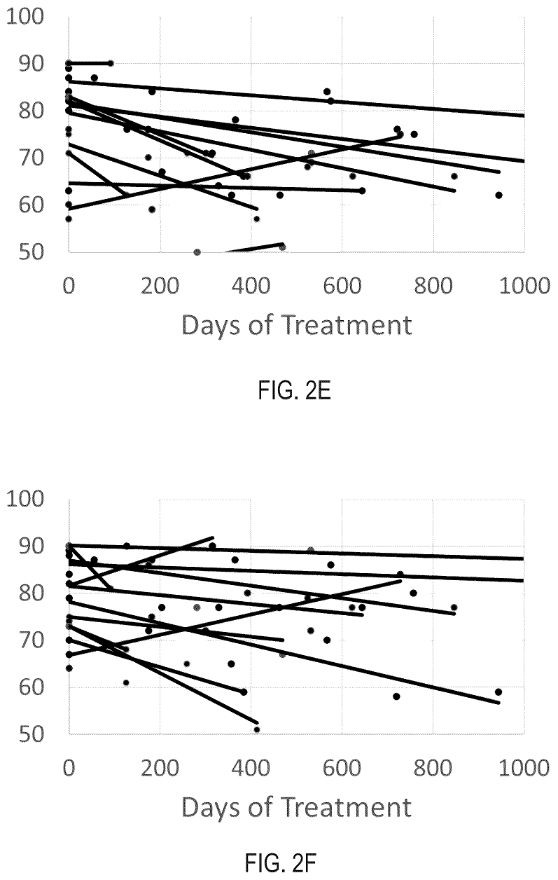

Turning now to FIGS. 2A-F, six figures indicate results of treatments when using the process (100) of FIG. 1. In sFBP positive human subjects (patients) started on the standard dose of leucovorin (25 mg twice a day) but in 6 of 12 (50%), the dose of leucovorin was increased to 50 mg twice a day in order to obtain an effective response as judged by the ongoing clinical evaluation. The number of days treated with leucovorin ranged from 92 to 1022 days with an average (SD) number of 584 (333) days of treatment. The graphs of FIGS. 2A-F illustrate the patients' responses using a Social Responsiveness Scale (SRS, which is known in the field, so will not be explained in detail herein).

The difference in SRS scores was compared before and after the start of leucovorin treatment. The Total score decreased by 7.4 (1.4) t-score points with leucovorin treatment [F(1,44.6)=28.03, p<0.001, Cohen's d′=1.4]. The Awareness subscale decreased by 8.0 (1.9) t-score points with leucovorin treatment [F(1,45.9)=18.01, p<0.001, Cohen's d′=1.1]. The Cognition subscale decreased by 8.5 (1.8) t-score points with leucovorin treatment [F(1,45.4)=21.64, p<0.001, Cohen's d′=1.2]. The Communication subscale decreased by 8.9 (1.6) t-score points with leucovorin treatment [F(1,44.6)=31.22, p<0.001, Cohen's d′=1.4]. The Motivation subscale decreased by 6.9 (2.1) t-score points with leucovorin treatment [F(1,44.8)=10.91, p<0.01, Cohen's d′=0.9]. The Mannerisms subscale decreased by 4.3 (1.9) t-score points with leucovorin treatment [F(1,45.0)=2.31, p=0.03, Cohen's d′=0.6].

To determine an estimate of the timing of the effect of leucovorin treatment, linear regression models were used to determine the change in SRS scores with each day of leucovorin treatment, as shown in FIGS. 2A-F. The Total score decreased by 0.01 (0.002) t-score points per day on leucovorin [F(1,46.6)=25.68, p<0.001]. The Awareness subscale decreased by 0.014 (0.003) t-score points per day on leucovorin [F(1,47.2)=19.74, p<0.001]. The Cognition subscale decreased by 0.013 (0.003) t-score points per day on leucovorin [F(1,47.4)=17.49, p<0.001]. The Communication subscale decreased by 0.013 (0.003) t-score points per day on leucovorin [F(1,46.2)=21.48, p<0.001]. The Motivation subscale decreased by 0.01 (0.004) t-score points per day on leucovorin [F(1,46.1)=8.27, p<0.01]. The Mannerisms subscale decreased by 0.008 (0.003) t-score points per day on leucovorin [F(1,46.2)=6.34, p=0.02].

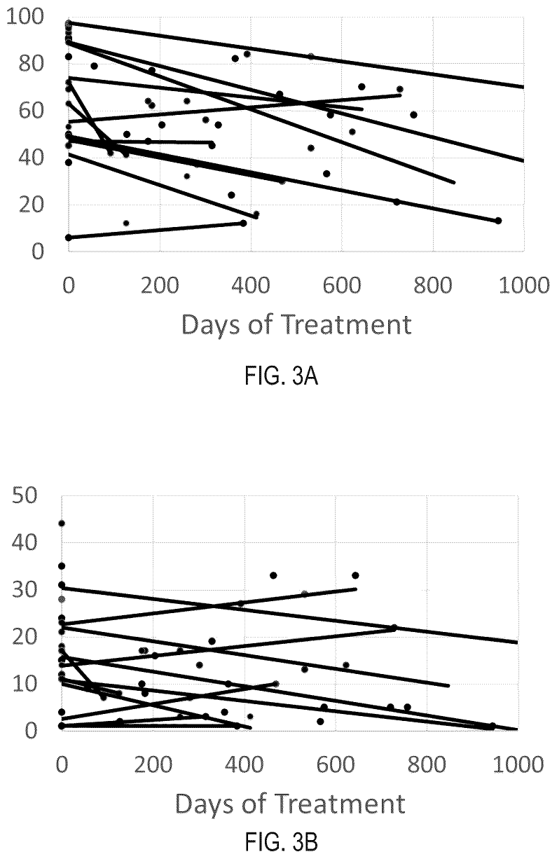

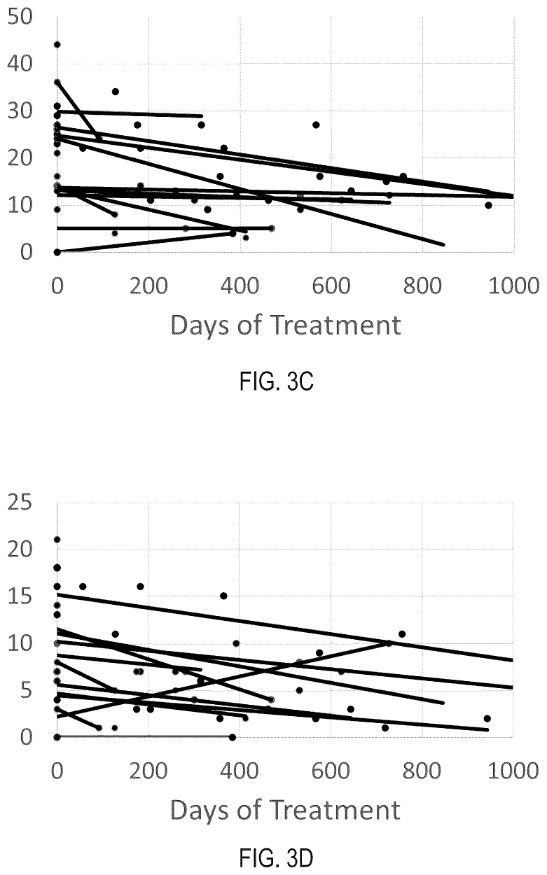

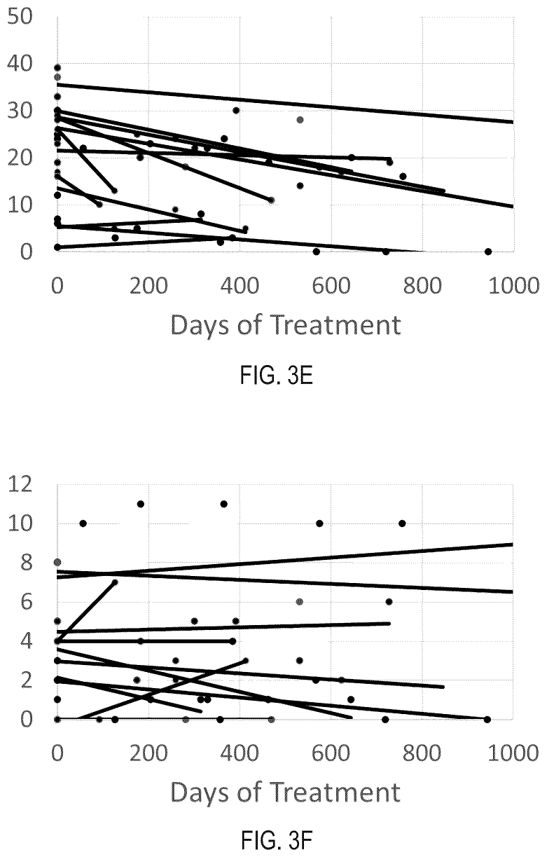

FIGS. 3A-F includes six charts that indicate results of treatments when using the process (100) of FIG. 1 using the Aberrant Behavioral Checklist (ABC, which is known in the field, so will not be explained in detail herein). The patients are the same as the patients in FIGS. 2A-F. Note that the scores/scales are different in FIGS. 3A-F, but the patients are the same and measured over the same time period as FIGS. 2A-F.

The difference in ABC scores was compared before and after the start of leucovorin treatment. The Total score decreased by 20.2 (5.9) raw points with leucovorin treatment [F(1,44.8)=11.92, p=0.001, Cohen's d′=0.9]. The Irritability subscale decreased by 5.7 (2.1) raw points with leucovorin treatment [F(1,44.8)=7.56, p<0.01, Cohen's d′=0.6]. The Social Withdrawal subscale decreased by 5.8 (1.7) raw points per day with leucovorin treatment [F(1,43.1)=11.05, p<0.01, Cohen's d′=0.9]. The Stereotypy subscale decreased by 2.2 (1.0) raw points with leucovorin treatment [F(1,46.4)=4.56, p<0.05, Cohen's d′=0.5]. The Hyperactivity subscale decreased by 6.8 (1.6) raw points with leucovorin treatment [F(1,43.8)=18.34, p<0.001, Cohen's d′=1.1]. Inappropriate Speech subscale did not change significantly with leucovorin treatment.

To get an estimate of the timing of the effect of leucovorin treatment, linear regression models were used to determine the change in ABC scores with each day of leucovorin treatment, as shown in FIGS. 3A-F. The Total score decreased by 0.04 (0.01) raw points per day on leucovorin [F(1,46.3)=12.67, p=0.001]. The Irritability subscale decreased by 0.008 (0.004) raw points per day on leucovorin [F(1,46.5)=5.33, p<0.05]. The Social-Withdrawal subscale decreased by 0.01 (0.003) raw points per day on leucovorin [F(1,44.9)=13.85, p<0.001]. The Stereotypy subscale decreased by 0.004 (0.002) raw points per day on leucovorin [F(1,48.1)=6.17, p<0.05]. The Hyperactivity subscale decreased by 0.01 (0.003) raw points per day on leucovorin [F(1,44.6)=20.56, p<0.001]. The change in the Inappropriate Speech subscale did not change significantly with leucovorin treatment.

Therefore, as can be seen in FIGS. 2A-3F, the process of FIG. 1 can be used to effectively treat patients that include sFBPs. Despite significant research, few validated biomarkers have been developed to assist in the diagnosis of ASD or for determining treatment response. Biomarkers of folate one-carbon metabolism are some of the most promising biomarkers for ASD, and FRAAs are one of the most promising biomarkers for determining treatment response. These studies emphasize the importance of the folate pathway in ASD and point to the promise of better understanding the specific abnormalities in folate one-carbon metabolism that can predispose children to developing ASD.

The folate pathway is also critical in other neurodevelopmental disorders such as neural tube defects and folate appears to be central to other diseases such as cancer. However, unlike neurodevelopmental disorders, cancer treatments aim to inhibit folate metabolism rather than to enhance it. sFBPs have been studied in cancer where they may have therapeutic effects. While the effect of inhibiting folate metabolism in cancer may be therapeutic, it may be detrimental for the developing brain. Thus, these sFBPs may be particularly important in understanding the risk of both developing neurodevelopmental disorders and in planning treatment. Indeed, half of the participants were prescribed higher than usual doses of leucovorin in order to obtain an obvious therapeutic effect. Thus, this suggests that sFBPs may significantly disrupt folate metabolism in children with ASD, requiring more aggressive treatment.

As discussed above, patients appeared to respond to treatment with leucovorin. The analysis suggests that treatment with leucovorin in these patients decreased the SRS score by about 7 points, a change that could potentially move the severity of the child from one category (severe, moderate, mild) to the next lower category. The decrease of the irritability subscale of the ABC was, on average, 5.7, which is an improvement consistent with the effect of anti-psychotic medications in well controlled clinical trials.

The analysis above demonstrated steady, but slow, improvements with leucovorin treatment in individuals positive for sFBPs. For example, a 5.7 improvement in irritability on the ABC generally requires two years of treatment rather than the typical 12-week trials used to examine improvement with anti-psychotic medications. Similarly, two years of treatment appear to be necessary to find the 7-point improvement in the SRS. However, it should be emphasized that few other treatments are effective in improving the SRS in individuals with ASD. Additionally, leucovorin is extremely safe and well-tolerated in this vulnerable population whereas anti-psychotic medication can have serious long-term adverse effects.

In other tests, many patients with ASD were tested for FRAAs and sFBPs to guide clinical management by identifying the clinical characteristics associated with these biomarkers and to determine whether the biomarkers could predict treatment response. For most scales of the SRS, a change in the scores when treated with leucovorin was dependent on the binding FRAA titer such that higher titers were associated with more severe ASD symptoms when not treated and less severe ASD symptoms when treated with leucovorin. Thus, it has been found that the change (for good) in symptoms is proportional to the FRAA titer. For the ABC scales, leucovorin only improved Irritability, but this treatment response was not related to folate biomarkers. Interestingly, higher binding FRAA titers were associated with worse Irritability. Treatment with mB12 Subcutaneous (Methyl-cobalamin) and/or fatty acids were associated with better SRS scores on a few subscales, fatty acids were associated with better ABC scores on two subscales and carnitine was associated with a better score on ABC Hyperactivity. In contrast, beta-blockers were associated with worse scores on most SRS subscales and ABC Irritability.

Patients with sFBPs appear to be more symptomatic and more complex than other ASD patients. Moreover, patients with sFBPs have more severe ASD symptoms as compared to those without sFBPs by about 5-6 points on the SRS scale, an amount that could easily push them into a higher level of ASD severity.

Recently, it was found that that the binding FRAA predicted response to leucovorin treatment for 5 of the 6 SRS scales. The SRS measures ASD symptoms, suggesting that ASD symptom improvement with leucovorin treatment is related to the binding FRAA titers. This is due to two opposing effects. It appears that those with higher binding FRAAs that were not treated with leucovorin had more severe ASD symptoms than those with low binding FRAA titers. In contrast, those with higher FRAA titers who were treated with leucovorin appear to have less severe ASD symptoms than those that were treated and had lower binding FRAA titers. Thus, overall, those with high FRAA titers had a greater response to leucovorin both because they were relatively more severe without treatment and relatively less severe with treatment. Those patients with some of the highest binding FRAA titers can have, on average, a eight-to-sixteen point favorable response on SRS subscales, an amount that can move them into more favorable severity categories.

The binding FRAA titer was found to be associated with increased Irritability, and treatment with leucovorin improved Irritability. This relationship with Irritability may be important, as the medications indicated for Irritability in ASD are antipsychotic medications which can have both short-term and long-term adverse effects.

There are few treatments that improve symptoms of ASD, but leucovorin improves core ASD symptoms. Leucovorin is an extremely safe and well-tolerated treatment, which is important in this vulnerable population. Few validated biomarkers are available to predict treatment response or assist with diagnosis in ASD. Studies have suggested that biomarkers of folate one-carbon metabolism are very promising to assist with ASD diagnosis and FRAAs are promising biomarkers to predict treatment response, which highlights a significance of the folate pathway in ASD. Several other treatments influenced core ASD symptoms as well as disruptive behaviors. Omega-3 fatty acids improved SRS social Awareness, Cognition, Communication, and Motivation as well as ABC Social, Withdrawal, and Stereotypy. Further, Methyl-cobalamin (mB12) was found to improve the SRS Total score, as well as all subscales except SRS Awareness.

Carnitine was found to improve ABC Hyperactivity. Carnitine has been found to improve core ASD symptoms in two DBPC studies and ABC Hyperactivity when combined with risperidone. Carnitine may be particularly important to supplement in individuals with ASD since, as a group, carnitine is low in many children with ASD and a subset of children with ASD may have a genetic deficiency in carnitine production.

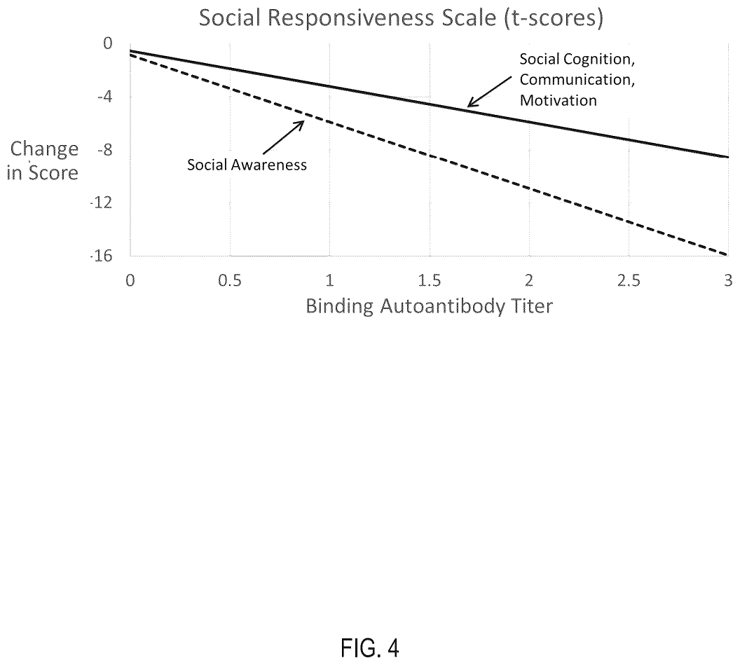

In general, Social Responsiveness Scale (SRS) t-scores are better (lower scores) as binding folate receptor alpha autoantibody titers increase with leucovorin treatment, while SRS scores are worse (higher scores) as binding folate receptor alpha autoantibody titers increase without leucovorin treatment. Moreover, Aberrant behavior checklist Irritability scores worsen as binding folate receptor alpha autoantibody titers increase, with leucovorin overall improving irritability. Thus, it has been found that using a FRAA titer (continuous analog reading) has been found to work better than looking just for a presence of FRAA (binary reading) to determine a treatment for people with ASD symptoms, as summarized in FIG. 4 (lower is better).

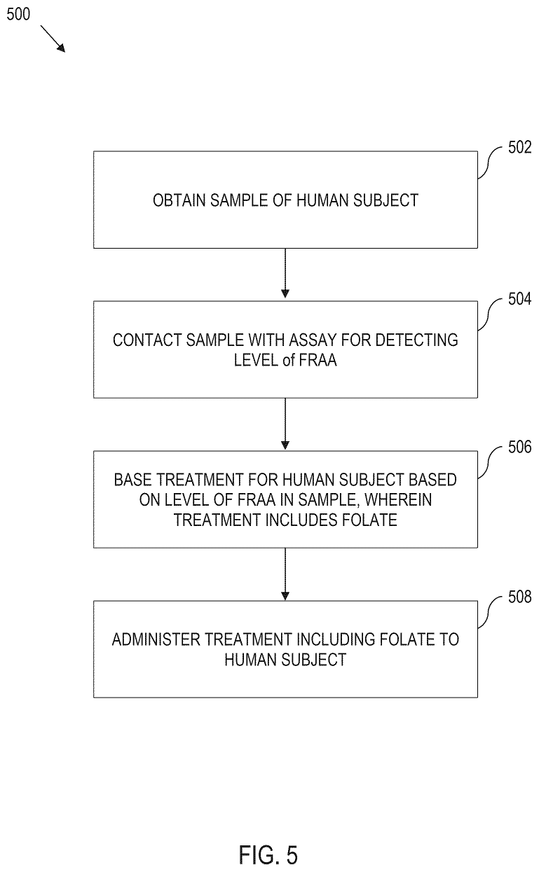

Turning now to FIG. 5, a process 500 for treating a human subject with a neurological disorder is shown. As discussed above, a neurologic disorder includes neurodevelopmental disorders, neurobehavioral disorders, and psychiatric disorders. For example, a neurologic disorder includes autism spectrum disorder (ASD). At 502, a sample is obtained from the human subject. The sample may be a blood serum sample or a saliva sample. Any method for obtaining the sample may be used (e.g., phlebotomy, receiving the serum sample previously drawn, sputum, etc.).

Once the sample is obtained, at 504, the sample is contacted with an assay for detecting a level of folate receptor alpha autoantibody (FRAA).

In many embodiments, the assay (or a different assay) also detects for a presence of sFBP and/or SNPs in the serum. As discussed above, the assay for detecting the and/or SNPs may give an indication of a mere presence of and/or SNPs, may give an indication of whether a level of and/or SNPs is above a threshold, or may give an indication of a level of and/or SNPs in the sample. In many embodiments, the assay can detect several different SNPs. For example, the assay can detect for RFC, FOLR1, BHMT, MTHFD2, DNMT, or combinations thereof. Thus, in these embodiments, five different SNPs may be used.

The types of assays discussed above may be used in any combination. Further, one assay may detect FRAA, sFBP, and SNPs. On the other hand, three (or more) different assays may be used. Alternatively, one assay may detect two of the biomarkers and a second assay detects the third biomarker.

At 506, a treatment for the human subject is developed based on a level of FRAA. A folate such as leucovorin (also called folinic acid) is used as at least part of the treatment. See above for more on folates.

The folate may be in any desired form. However, at least one variable of the treatment is based on the level of FRAA found in the sample. For example, the treatment may include an X-dosage of folate given Y times per day with a time release of Z minutes. The dosage of folate may be linearly related (e.g., proportionally) to the level of FRAA found in the sample, geometrically related to the level of FRAA found in the sample, etc.

In embodiments that also look for sFBP and/or SNPs, the presence and/or levels of sFBP and/or SNPs also guide the variables for creating a treatment of folate for the human subject. For example, if a sample includes RFC, FOLR1, BHMT, or combinations thereof, then a higher dose of folate may be used. However, if MTHFD2 or DNMT is present, then the folate dosage may be lower. In several embodiments, mitochondrial supplements (e.g., carnitine) are added to the dosage of folate. In numerous embodiments, omega-3 fatty acids are added to the dosage of folate. The addition of mitochondrial supplements and/or omega-3 fatty acids have been shown to improve response of the human subject with respect to the treatment.

One of the objectives that the treatment may have is to enhance folate metabolism within the human subject.

At 508, the treatment is administered to the human subject.

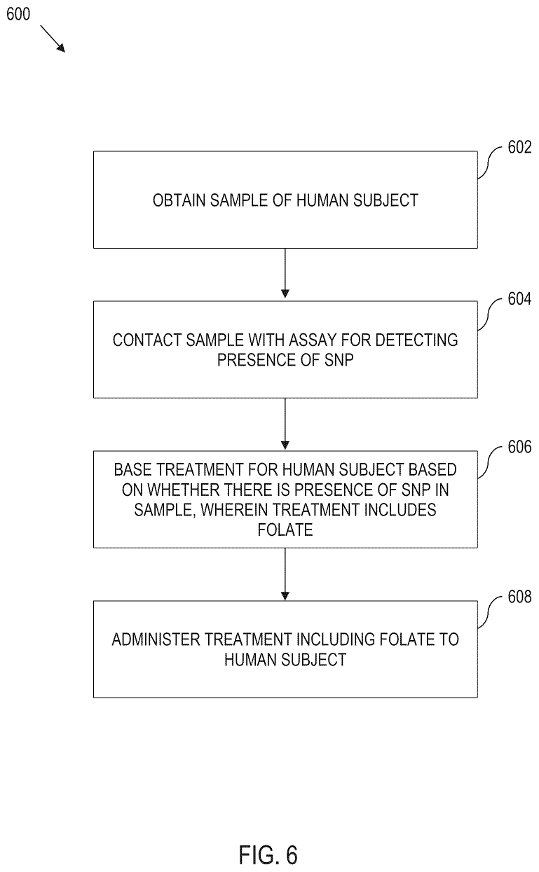

Turning now to FIG. 6, a process 600 for treating a human subject with a neurological disorder is shown. As discussed above, a neurologic disorder includes neurodevelopmental disorders, neurobehavioral disorders, and psychiatric disorders. For example, a neurologic disorder includes autism spectrum disorder (ASD). At 602, a sample is obtained from the human subject. The sample may be a blood serum sample or a saliva sample. Any method for obtaining the sample may be used (e.g., phlebotomy, receiving the serum sample previously drawn, sputum, etc.).

Once the sample is obtained, at 604, the sample is contacted with an assay for detecting a presence of a single nucleotide polymorphism (SNP) in folate and related one-carbon metabolism genes. In some embodiments, the assay for detecting the presence of SNPs merely gives an indication of whether a SNP is present in the sample. In various embodiments, the assay for detecting the presence of SNPs gives an indication of whether one or more SNPs is present in the sample is above a predetermined threshold. In numerous embodiments, the assay for detecting the presence of SNPs detects a level of one or more SNPs in the sample and gives an indication of the level of one or more SNPs.

In many embodiments, the assay can detect several different SNPs. For example, the assay can detect for RFC, FOLR1, BHMT, MTHFD2, DNMT, or combinations thereof. Thus, in these embodiments, five different SNPs may be used.

In several embodiments, the assay (or a different assay) also detects a presence of a folate receptor alpha autoantibody (FRAA). As discussed above, the assay for detecting the FRAA may give an indication of a mere presence of FRAA, may give an indication of whether a level of FRAA is above a threshold, or may give an indication of a level of FRAA in the sample.

In many embodiments, the assay (or a different assay) also detects for a presence of sFBP in the serum. As discussed above, the assay for detecting the sFBP may give an indication of a mere presence of sFBP, may give an indication of whether a level of sFBP is above a threshold, or may give an indication of a level of sFBP in the sample.

The three types of assays discussed above may be used in any combination. Further, one assay may detect sFBP, FRAA, and SNPs. On the other hand, three (or more) different assays may be used. Alternatively, one assay may detect two of the biomarkers and a second assay detects the third biomarker.

At 606, a treatment for the human subject is developed based on whether there is a presence of one or more SNPs in the sample, a level of FRAA, or both. A folate such as leucovorin (also called folinic acid) is used as at least part of the treatment. See above for more on folates.

The folate may be in any desired form. However, at least one variable of the treatment is based on the one or more SNPs found in the sample. For example, the treatment may include an X-dosage of folate given Y times per day with a time release of Z minutes. In embodiments that detect a level of one or more SNP in the sample, the dosage of folate may be linearly related (e.g., proportionally) to the level of the one or more SNP, geometrically related to the level of the one or more SNP, different level ranges may have different mathematical formulae, etc.

In embodiments that also look for sFBP and/or FRAA, the presence and/or levels of sFBP and/or FRAA also guide the variables for creating a treatment of folate for the human subject. For example, if a sample includes RFC, FOLR1, BHMT, or combinations thereof, and FRAA (binding or blocking), then a higher dose of folate may be used. As another example, the titer of the binding FRAA (whether in conjunction with RFC and/or FOLR1) may indicate a higher dosage. However, if MTHFD2 or DNMT is present, then the folate dosage may be lower. In several embodiments, mitochondrial supplements (e.g., carnitine) are added to the dosage of folate. In numerous embodiments, omega-3 fatty acids are added to the dosage of folate. The addition of mitochondrial supplements and/or omega-3 fatty acids have been shown to improve response of the human subject with respect to the treatment.

One of the objectives that the treatment may have is to enhance folate metabolism within the human subject.

At 608, the treatment is administered to the human subject.

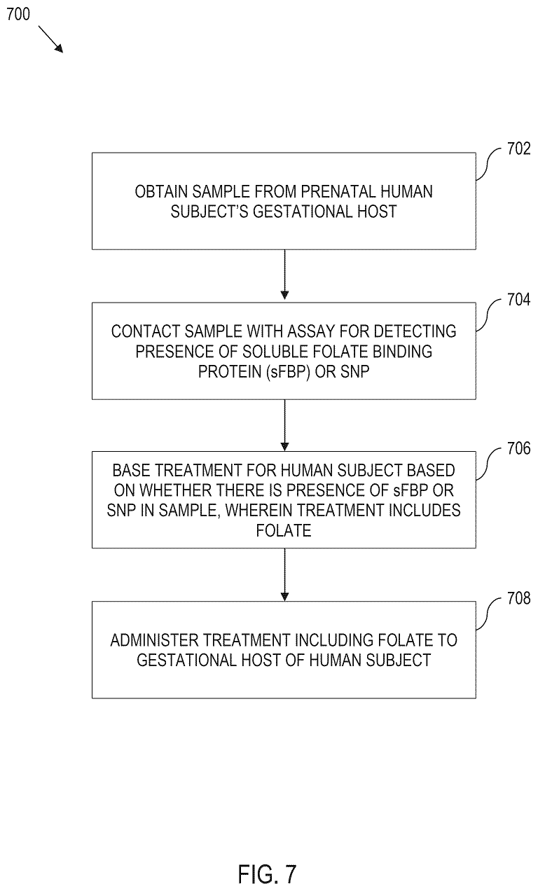

FIG. 7 illustrates a process 700 for treating a prenatal human subject with a neurological disorder. At 702, a sample is obtained from the prenatal human subject's gestational host. As used herein, a gestational host is a body that a prenatal human being (fetus) is growing. For most fetuses, the gestational host will be the fetus's biological mother. However, in some instances the gestational host may be a surrogate carrying the fetus to term. Any method for obtaining the sample may be used (e.g., phlebotomy, receiving the sample previously drawn, sputum etc.). At 704, the sample is contacted with an assay for detecting sFBP and/or one or more SNPs. If the assay is for sFBP, then 704 and 706 are similar to 104 and 106, as discussed in FIG. 1. If the assay is for one or more SNPs, then 704 and 706 are similar to 604 and 606, as discussed in FIG. 6. At 708, the treatment is administered to the human subject's gestational host.

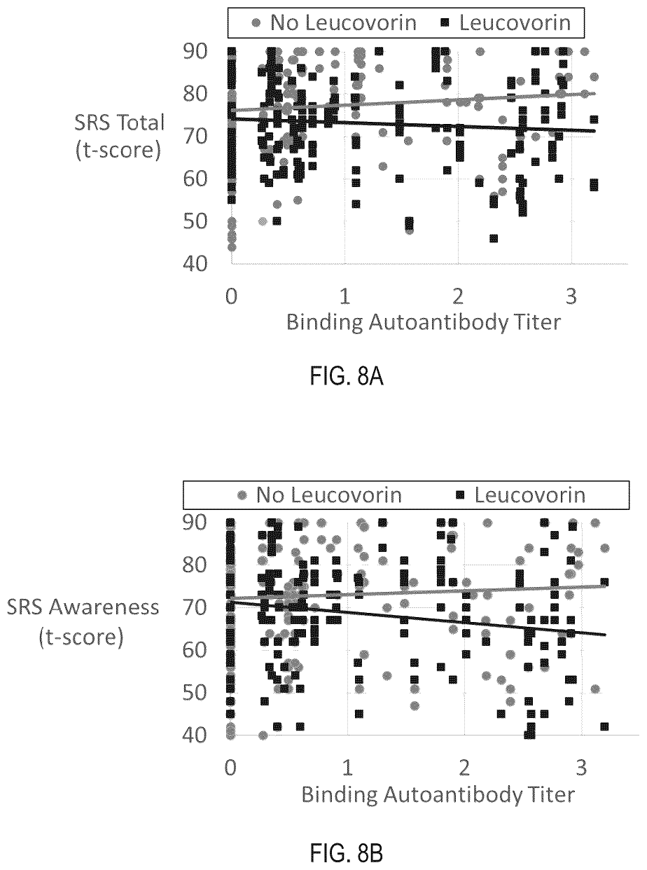

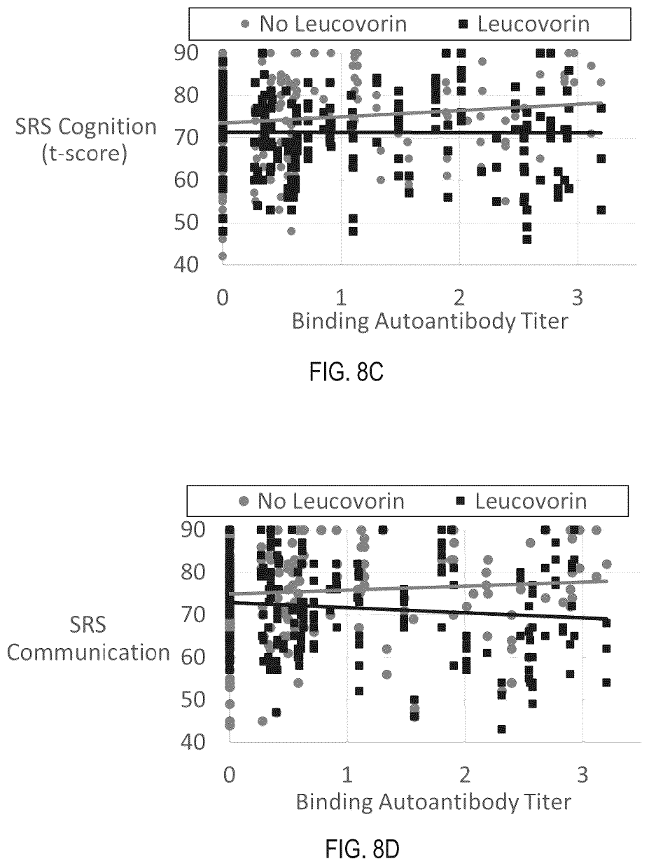

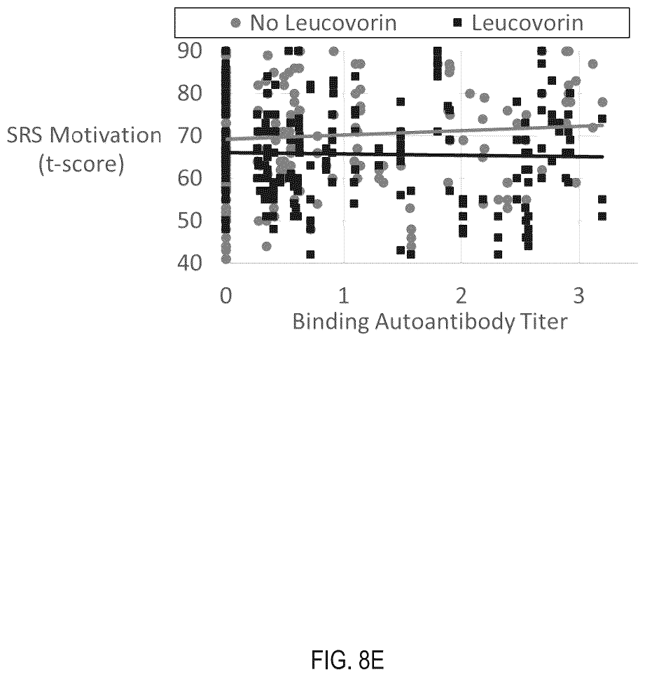

FIGS. 8A-E are a collection of five charts illustrating results of factoring in a titer of binding autoantibody with the folate treatment when applied to several patients, where the results are shown on a Social Responsiveness Scale. Social Responsiveness Scale (SRS) t-scores are better (lower scores) as binding folate receptor alpha autoantibody titers increase with leucovorin treatment (orange points and line) while SRS score are worse (higher scores) as binding folate receptor alpha autoantibody titers increase without leucovorin treatment (blue points and line).

The following two tables indicate some treatments and a statistically significant effect of concurrent treatments. β indicates an effectiveness on a certain outcome measure. In Table 2 and 3, a negative value for beta indicates a positive result.

TABLE 2
Effect of Concurrent Treatments on Social Responsiveness Scale

| Outcome | mB12 SQ | Fatty Acids | Carnitine |
|---|---|---|---|
| Measure | β | β | β |
| Total | −2.8 (1.1)** | | |
| Cognition | −4.3 (1.3)*** | −2.7 (1.2)* | |
| Communication | | −2.7 (1.2)* | |
| Motivation | −3.2 (1.3)* | −2.9 (1.3)* | −2.3 (1.0)* |

(*p < 0.05; **p < 0.01; ***p < 0.001)

TABLE 3
Effect of Concurrent Treatments on Aberrant Behavior Checklist

| Outcome | Oral B12 | Fatty Acids |
|---|---|---|
| Measure | β | β |
| Social Withdrawal | | −1.9 (0.9)* |
| Stereotypy | −0.9 (0.4)* | −1.2 (0.5)* |
| Hyperactivity | | −2.5 (1.1)* |

(*p < 0.05; **p < 0.01; ***p < 0.001)

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the invention in the form disclosed. Many modifications and variations will be apparent to those of ordinary skill in the art without departing from the scope and spirit of the invention. Aspects of the disclosure were chosen and described in order to best explain the principles of the invention and the practical application, and to enable others of ordinary skill in the art to understand the invention for various embodiments with various modifications as are suited to the particular use contemplated.

What is claimed is:

1. A process for treating a human subject with a neurologic disorder, the process comprising:
obtaining a serum sample of the human subject;
contacting the sample with an assay for detecting a presence of soluble folate binding protein (sFBP);
basing a treatment for the human subject based on the whether there is a presence of sFBP in the sample, wherein the treatment includes a folate; and
administering the treatment including the folate to the human subject.

2. The process of claim 1, wherein basing a treatment for the human subject based on the whether there is a presence of sFBP in the sample comprises using a higher dosage of the folate if there is a presence of sFBP in the sample.

3. The process of claim 1, further comprising:
Contacting the sample with an assay for detecting a presence of one or more single nucleotide polymorphism (SNP) in folate or related one-carbon metabolism genes;
wherein basing a treatment for the human subject further comprises basing the treatment for the human subject based on the whether there is a presence of the SNP in the sample.

4. The process of claim 3, wherein:
contacting the sample with an assay for detecting a presence of one or more single nucleotide polymorphism (SNP) in folate or related one-carbon metabolism genes comprises contacting the sample with an assay for detecting a presence of RFC, FOLR1, or BHMT; and
basing a treatment for the human subject further comprises basing the treatment for the human subject by having a higher dosage of folate if RFC, FOLR1, or BHMT is in the sample.

5. The process of claim 3, further comprising:
contacting the sample with an assay for detecting a presence of one or more single nucleotide polymorphism (SNP) in folate or related one-carbon metabolism genes comprises contacting the sample with an assay for detecting a presence of MTHFD2 or DNMT; and
basing a treatment for the human subject further comprises basing the treatment for the human subject by having a lower dosage of folate if MTHFD2 or DNMT is in the sample.

6. The process of claim 1, further comprising:
contacting the sample with an assay for detecting a presence of a folate receptor alpha autoantibody;
wherein basing a treatment for the human subject further comprises basing the treatment for the human subject based on an amount of folate receptor alpha autoantibody in the sample.

7. The process of claim 1, wherein:
contacting the sample with an assay for detecting a presence of sFBP further comprises contacting the sample with the assay for detecting a level of sFBP within the sample; and
basing a treatment for the human subject further comprises basing a treatment for the human subject based on the level of sFBP in the sample.

8. The process of claim 7, wherein:
basing the treatment for the human subject based on the level of sFBP in the sample comprises determining an amount of folate to provide to the human subject based proportionally on the level of sFBP in the sample.

9. The process of claim 7, wherein:
basing the treatment for the human subject based on the level of sFBP in the sample comprises determining an amount of folate to provide to the human subject based on a mathematical formula that uses the level of sFBP in the sample as a variable.

10. The process of claim 1, wherein:
basing a treatment for the human subject further includes enhancing folate metabolism.

11. A process for treating a human subject with a neurologic disorder, the process comprising:
obtaining a sample of the human subject;
contacting the sample with an assay for detecting a presence of one or more single nucleotide polymorphism (SNP) in folate genes;
basing a treatment for the human subject based on the whether there is a presence of a SNP in the sample, wherein the treatment includes a folate; and
administering the treatment including the folate to the human subject.

12. The process of claim 11, wherein:
contacting the sample with an assay for detecting a presence of one or more single nucleotide polymorphism (SNP) in folate or related one-carbon metabolism genes comprises contacting the sample with an assay for detecting a presence of RFC, FOLR1, or BHMT; and
basing a treatment for the human subject further comprises basing the treatment for the human subject by having a higher dosage of folate if RFC or FOLR1 is in the sample.

13. The process of claim 12, further comprising:
contacting the sample with an assay for detecting a presence of one or more single nucleotide polymorphism (SNP) in folate or related one-carbon metabolism genes comprises contacting the sample with an assay for detecting a presence of MTHFD2 or DNMT; and
basing a treatment for the human subject further comprises basing the treatment for the human subject by having a lower dosage of folate if MTHFD2 or DNMT is in the sample.

14. The process of claim 11, further comprising:
contacting the sample with an assay for detecting a presence of one or more single nucleotide polymorphism (SNP) in folate or related one-carbon metabolism genes comprises contacting the sample with an assay for detecting a presence of MTHFD2 or DNMT; and
basing a treatment for the human subject further comprises basing the treatment for the human subject by having a lower dosage of folate if MTHFD2 or DNMT is in the sample.

15. The process of claim 1, wherein:
contacting the sample with an assay for detecting a presence of one or more single nucleotide polymorphism (SNP) in folate or related one-carbon metabolism genes further comprises contacting the sample with an assay for detecting a presence of soluble folate binding protein (sFBP); and
basing a treatment for the human subject based on the whether there is a presence of sFBP in the sample comprises using a higher dosage of the folate if there is a presence of sFBP in the sample.

16. The process of claim 1, further comprising:
contacting the sample with an assay for detecting an amount of a folate receptor alpha autoantibody;
wherein basing a treatment for the human subject further comprises basing the treatment for the human subject based on an amount of folate receptor alpha autoantibody in the sample.

17. The process of claim 1, wherein:
contacting the sample with an assay for detecting a presence of one or more SNPs further comprises contacting the sample with the assay for detecting a level of the one or more SNPs within the sample; and
basing a treatment for the human subject further comprises basing a treatment for the human subject based on the level of the one or more SNPs in the sample.

18. The process of claim 17, wherein:
obtaining a sample of the human subject comprises obtaining a blood serum sample from the human subject.

19. The process of claim 17, wherein:
obtaining a sample of the human subject comprises obtaining a saliva sample from the human subject.

20. A process for detecting a potential for a neurological disorder in a prenatal human subject, the process comprising:
obtaining a sample from the prenatal human subject's gestational host;
contacting the sample with an assay for detecting a presence of soluble folate binding protein (sFBP) or one or more single nucleotide polymorphism (SNP) in folate ore related one-carbon metabolism genes;
basing a treatment for the human subject based on the whether there is a presence of sFBP or the one or more SNPs in the sample, wherein the treatment includes a folate; and
administering the treatment including the folate to the gestational host of the human subject.
